(19) United States
(12) Patent Application Publication
(10) Pub. No.: US 2017/0364069 A1
(43) Pub. Date: Dec. 21, 2017

(54) AUTONOMOUS BEHAVIORAL OVERRIDE UTILIZING AN EMERGENCY CORRIDOR

(71) Applicant: Ford Global Technologies, LLC, Dearborn, MI (US)

(72) Inventors: Nicholas Colella, Grosse Ile, MI (US); Stephen Jay Orris, JR., New Boston, MI (US); David A. Herman, Southfield, MI (US); Nicholas Alexander Scheufler, Flat Rock, MI (US); Nunzio DeCia, Northville, MI (US)

(21) Appl. No.: 15/184,487

(22) Filed: Jun. 16, 2016

(51) Int. Cl. G05D 1/00; A61B 5/00; G05D 1/02; H04W 4/22; H04W 4/02

(52) U.S. Cl. CPC G05D 1/0061; H04W 4/22; H04W 4/02; G05D 1/0276; A61B 5/747; G05D 1/0088; G05D 1/0022; G05D 1/0212

(57) ABSTRACT

Systems and methods are disclosed for autonomous behavior override utilizing an emergency corridor. An example disclosed system includes an autonomous vehicle, infrastructure nodes, and an emergency router. The example autonomous vehicle sends an emergency request in response to detecting an occupant experiencing a medical emergency. The example infrastructure nodes are distributed across a municipal area. The example emergency router selects a route from a first location of the autonomous vehicle to an emergency response facility. The example emergency router also selects the infrastructure nodes that are along the route. Additionally, the example emergency router instructs the selected infrastructure nodes to broadcast messages to create an emergency corridor.

### TECHNICAL FIELD

The present disclosure generally relates to autonomous vehicles and, more specifically, autonomous behavioral override utilizing an emergency corridor.

### BACKGROUND

Autonomous vehicles use sensors to navigate without driver input. Generally, autonomous vehicles are designed to follow laws. In fact, autonomous vehicles have been involved in incidents with other vehicles because the autonomous vehicle followed the law and the human driver did not.

### SUMMARY

The appended claims define this application. The present disclosure summarizes aspects of the embodiments and should not be used to limit the claims. Other implementations are contemplated in accordance with the techniques described herein, as will be apparent to one having ordinary skill in the art upon examination of the following drawings and detailed description, and these implementations are intended to be within the scope of this application.

Example embodiments are disclosed for autonomous behavior override utilizing an emergency corridor. An example disclosed system includes an autonomous vehicle, infrastructure nodes, and an emergency router. The example autonomous vehicle sends an emergency request in response to detecting an occupant experiencing a medical emergency. The example infrastructure nodes are distributed across a municipal area. The example emergency router selects a route from a first location of the autonomous vehicle to an emergency response facility. The example emergency router also selects the infrastructure nodes that are along the route. Additionally, the example emergency router instructs the selected infrastructure nodes to broadcast messages to create an emergency corridor.

An example disclosed method to create an emergency corridor for an autonomous vehicle includes receiving an emergency request from the autonomous vehicle. The example method also includes selecting an emergency facility. The example method includes determining a route from the autonomous vehicle to the emergency facility. Additionally, the example method includes determining infrastructure nodes along the route, and broadcasting emergency messages from the infrastructure nodes along the route. The emergency messages include the route, a location, a heading, and a speed of the autonomous vehicle.

An example disclosed method includes monitoring biometric sensors in an autonomous vehicle to detect when an occupant is experiencing a medical emergency. The example method also includes, in response to detecting the medical emergency, sending an emergency request to an emergency dispatch server. Additionally, in response to receiving instructions from the emergency dispatch server, operating the autonomous vehicle to follow a route specified by the instructions.

### BRIEF DESCRIPTION OF THE DRAWINGS

For a better understanding of the invention, reference may be made to embodiments shown in the following drawings. The components in the drawings are not necessarily to scale and related elements may be omitted, or in some instances proportions may have been exaggerated, so as to emphasize and clearly illustrate the novel features described herein. In addition, system components can be variously arranged, as known in the art. Further, in the drawings, like reference numerals designate corresponding parts throughout the several views.

FIG. 1 is a system diagram depicting a map with an emergency corridor in accordance with the teachings of this disclosure.

FIG. 2 is a block diagram of the emergency dispatch server of FIG. 1.

FIG. 3 is a block diagram of electronic components of the emergency dispatch server of FIGS. 1 and 2.

FIG. 4 is a block diagram of electronic components of the autonomous vehicle of FIG. 1.

FIG. 5 is a flowchart of an example method to override autonomous behavior of the vehicle of FIG. 1.

FIG. 6 is a flowchart of an example method to create the emergency corridor of FIG. 1.

FIG. 7 is a flowchart of an example method to broadcast emergency messages by infrastructure nodes along the emergency corridor.

FIG. 8 is a flowchart of an example method for the vehicles to react to the emergency messages broadcast by the infrastructure nodes along the emergency corridor.

### DETAILED DESCRIPTION OF EXAMPLE EMBODIMENTS

While the invention may be embodied in various forms, there are shown in the drawings, and will hereinafter be described, some exemplary and non-limiting embodiments, with the understanding that the present disclosure is to be considered an exemplification of the invention and is not intended to limit the invention to the specific embodiments illustrated.

Autonomous vehicles are configured to follow traffic laws. Under normal conditions, this is a desired feature. However, if an occupant of the autonomous vehicle is experiencing an emergency (e.g., has a medical problem), strict adherence to the law may not be as desirable. As disclosed below, the autonomous vehicle includes biometric sensors to detect when an occupant of the vehicle is experiencing an emergency (e.g., has a medical problem). Additionally, in some examples, the autonomous vehicle also includes an emergency request input (e.g., a physical button, a virtual button on a touch screen, etc.). When the autonomous vehicle detects that the occupant of the vehicle is experiencing an emergency and/or the emergency request input is activates, the vehicle sends an emergency request to an emergency dispatch server. The emergency dispatch server selects a medical facility (e.g., a hospital, a clinic, a triage center, etc.). Additionally, the emergency dispatch server may give the autonomous vehicle permission to engage in emergency maneuvers (e.g., speed, run red lights, etc.).

In municipal areas, it can be difficult to navigate through traffic. As disclosed below, an emergency corridor is created using dedicated short range communication (DSRC) nodes installed on infrastructure (e.g., traffic lights, traffic control boxes, buildings, lamp posts, bridges, tunnels, etc.). When the autonomous vehicle transmits an emergency request, an emergency router of the emergency dispatch server selects a corridor route from the current location of the autonomous vehicle to the medical facility. The corridor route is based on for example, weather data, traffic data, navigation data, and locations of nodes installed on the infrastructure (sometime referred to as “infrastructure nodes”). After the corridor route is selected, the emergency router instructs the infrastructure nodes along the corridor route to broadcast an emergency corridor message. The emergency corridor message includes information to inform other vehicles of the emergency corridor and instructions regarding how to behave. For example, the emergency corridor message may include the location of the autonomous vehicle, the velocity of the autonomous vehicle, the route of the emergency corridor, and a requested lane to move out of. The emergency dispatch server provides the route of the emergency corridor to the autonomous vehicle. If authorized by the emergency dispatch server, the autonomous vehicle traverses the emergency corridor using the emergency maneuvers. Otherwise, the autonomous vehicle traverses the emergency corridor using the standard autonomous maneuvers. In some examples, the autonomous vehicle will also broadcast the emergency corridor message.

The other vehicles that receive the emergency corridor message determine if their route will run parallel or intersect the emergency corridor. If so, the vehicle will present an audio and/or visual notification to the occupants of the vehicle and provide instructions (e.g., “pull over to the right”). In some examples, the vehicles that receiver the emergency corridor message rebroadcast the emergency corridor message. In such a manner, the emergency corridor message may be propagated in areas where the infrastructure nodes are sparse or in locations where the DSRC signals do not travel far (e.g., locations with tall buildings, etc.).

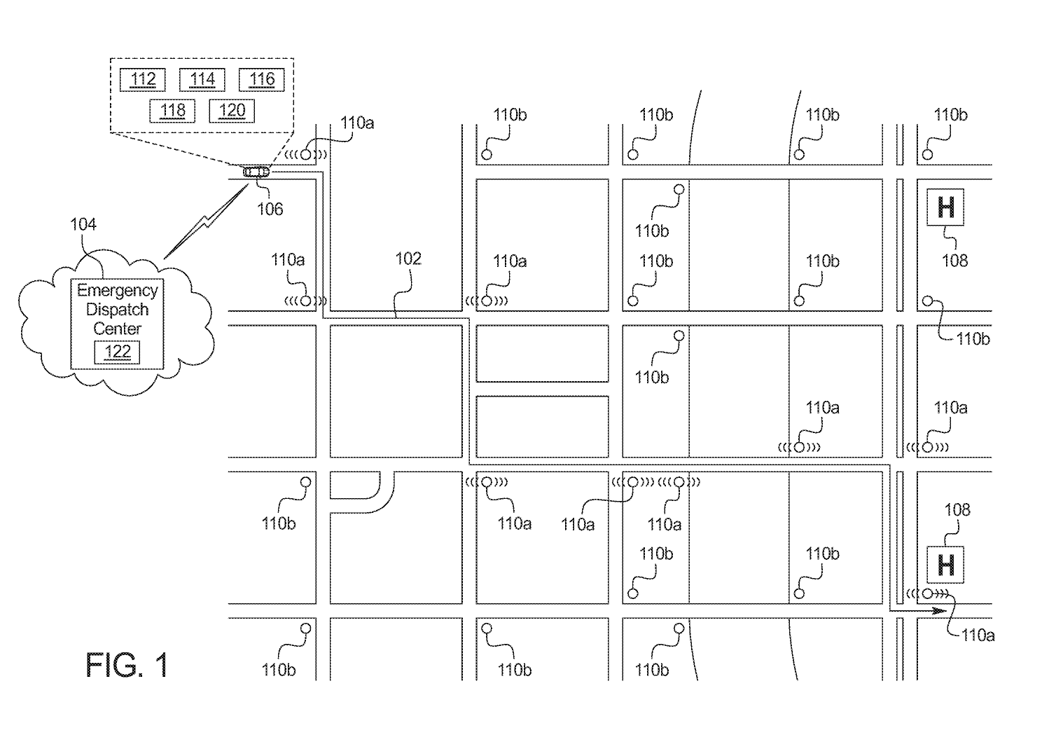

FIG. 1 is a system diagram depicting a map with an emergency corridor 102 in accordance with the teachings of this disclosure. From time-to-time, an emergency dispatch server 104 receives an emergency request from an autonomous vehicle 106. The autonomous vehicle 106 receives instructions from the emergency dispatch server 104 to travel to an emergency response facility 108 using the emergency corridor 102. In some examples, the instructions include an authorization to travel through the emergency corridor 102 in an emergency mode.

Infrastructure nodes 110a and 110b are installed on infrastructure around a municipal area. For example, the infrastructure nodes 110a and 110b may be installed on traffic signals, traffic control boxes, bridges, tunnel entrances, lamp posts, etc. The infrastructure nodes 110a and 110b are communicatively coupled to the emergency dispatch server 104. When instructed by the emergency dispatch server 104, the infrastructure nodes 110a along the emergency corridor 102 broadcast an emergency corridor message via direct short range communication (DSRC). In some examples, the infrastructure nodes 110a and 110b track the location of the autonomous vehicle 106 based on the emergency corridor messages. In such examples, the infrastructure nodes 110a along the route of the emergency corridor 102 stop broadcasting the emergency corridor messages when the autonomous vehicle 106 has passed the respective infrastructure node 110a.

The example infrastructure nodes 110a and 110b include antenna(s), radio(s) and software to broadcast the emergency corridor messages. DSRC is a wireless communication protocol or system, mainly meant for transportation, operating in a 5.9 GHz spectrum band. More information on the DSRC network and how the network may communicate with vehicle hardware and software is available in the U.S. Department of Transportation's Core June 2011 System Requirements Specification (SyRS) report (available at http://www.its.dot.gov/meetings/pdf/CoreSystem_SE_SyRS_RevA %20(2011-06-13).pdf), which is hereby incorporated by reference in its entirety along with all of the documents referenced on pages 11 to 14 of the SyRS report. DSRC systems may be installed on vehicles and along roadsides on infrastructure. DSRC systems incorporating infrastructure information is known as a “roadside” system. DSRC may be combined with other technologies, such as Global Position System (GPS), Visual Light Communications (VLC), Cellular Communications, and short range radar, facilitating the vehicles communicating their position, speed, heading, relative position to other objects and to exchange information with other vehicles or external computer systems. DSRC systems can be integrated with other systems such as mobile phones.

Currently, the DSRC network is identified under the DSRC abbreviation or name. However, other names are sometimes used, usually related to a Connected Vehicle program or the like. Most of these systems are either pure DSRC or a variation of the IEEE 802.11 wireless standard. The term DSRC will be used throughout herein. However, besides the pure DSRC system it is also meant to cover dedicated wireless communication systems between cars and roadside infrastructure system, which are integrated with GPS and are based on an IEEE 802.11 protocol for wireless local area networks (such as, 802.11p, etc.).

In the illustrated example, the autonomous vehicle 106 includes an autonomy unit 112 and a health monitoring unit 114. The autonomy unit 112 controls the operation of the autonomous vehicle 106 based on input from sensors (e.g., ultrasonic sensors, RADAR, LiDAR, cameras, etc.) and navigation data. The autonomy unit 112 includes two modes, a standard mode and an emergency mode. In the standard mode, the autonomy unit 112 operates the autonomous vehicle 106 in accordance standard traffic laws and regulations. For example, the autonomy unit 112 may observe the speed limit. In emergency mode, the autonomy unit 112 operates the autonomous vehicle 106 under a set of emergency laws and regulations (e.g., exceed the speed limit, ignore traffic signals, traveling the wrong way down a one-way street, turning on restrictive turns, traveling on a shoulder of a highway, etc.). For example, the autonomy unit 112 in emergency mode may exceed the speed limit long while traversing the emergency corridor 102. In some examples, the autonomy unit 112 is unable to enter the emergency mode until authorized by the emergency dispatch server 104.

The health monitoring unit 114 monitors a health status of occupants of the autonomous vehicle 106. To monitor the health status of occupants, the health monitoring unit 114 includes sensors distributed around the autonomous vehicle 106. The health monitoring unit 114 may include, for example, cameras, pulse sensors embedded in seats, carbon dioxide sensors, infrared sensors, etc. Additionally, in some examples, the health monitoring unit 114 is communicatively coupled to wearable devices that monitor the health status of the occupants with sensors integrated into the wearable device, such as a moisture sensor, a heartbeat sensor, a breathing sensor, a temperature sensor, a pulse oximeter, etc. The health monitoring unit 114 compares the data from the sensors to determine when one of the occupants of the autonomous vehicle is experiencing an emergency. In response to detecting an emergency, the health monitoring unit 114 sends the emergency request to the emergency dispatch server 104. Additionally, in some examples, the health monitoring unit 114 includes the data from the sensors in the emergency request to be forwarded to the emergency response facility 108.

The autonomous vehicle 106 is in communication with the emergency dispatch server 104. In some examples, the autonomous vehicle 106 is also equipped with a DSRC module 116 to communicate with the emergency dispatch server 104 via the infrastructure nodes 110a and 110b and to broadcast the emergency corridor messages. In some examples, the autonomous vehicle includes a cellular modem 118 to communicate with the emergency dispatch server 104. The cellular modem 118 connects to an external network (e.g., the Internet) using standards-based wide area networks (e.g., Global System for Mobile Communications (GSM), Universal Mobile Telecommunications System (UMTS), Long Term Evolution (LTE), Code Division Multiple Access (CDMA), WiMAX (IEEE 802.16m), and Wireless Gigabit (IEEE 802.11ad), etc.). Additionally, the autonomous vehicle 106 includes a global positioning system (GPS) receiver 120 to provide the coordinates of the autonomous vehicle 106 to the emergency dispatch server 104.

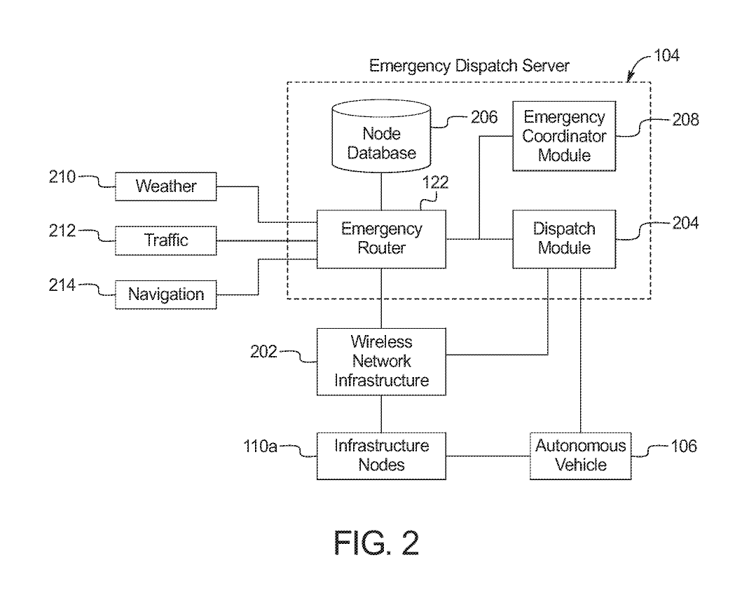

The emergency dispatch server 104 includes an emergency router 122 to generate the emergency corridor 102 based on the current location of the autonomous vehicle 106 and the emergency response facility 108. As disclosed in connection with FIG. 2 below, the emergency router 122 selects (a) the emergency response facility 108, and (b) a route for the emergency corridor 102. The emergency dispatch server 104 selects the emergency response facility 108 based on (i) the distance between potential emergency response facilities 108 and the autonomous vehicle 106, (ii) preferences of the occupants that is experiencing the emergency (e.g., from a profile stored by the autonomous vehicle 106 and/or the emergency dispatch server 104), (iii) estimated travel time to the potential emergency response facilities 108, (iv) the nature of the emergency, (v) specialties of the potential emergency response facilities 108, and/or (vi) availability of the potential emergency response facilities 108, etc.

The emergency router 122 informs the selected emergency response facility 108 regarding the incoming autonomous vehicle 106, an estimated time of arrival (ETA), and the nature of the emergency. In some examples, the emergency router 122 requests confirmation from the selected emergency response facility 108 that the autonomous vehicle 106 has arrived and that the occupant has been located. Additionally, in some examples, the emergency router 122 sends the health data included in the emergency request to the selected emergency response facility 108. In such a manner, the selected emergency response facility 108 may prepare for the arrival of the autonomous vehicle 106. In some examples, the emergency router 122 determines whether the emergency request was sent from the autonomous vehicle 106 in good faith. As used herein, the term “bad faith” is defined as sending an emergency request by pressing an emergency button and/or causing the health monitoring unit 114 to trigger an emergency request when none of the occupants are experiencing an emergency. In some examples, the emergency router 122 determines the emergency request was sent in bad faith if confirmation of arrival was not received from the selected emergency response facility 108. Additionally or alternatively, in some examples, the emergency router 122 determines emergency request was sent in bad faith if GPS coordinates of the autonomous vehicle 106 reveal that the autonomous vehicle 106 did not remain stationary for a threshold amount of time (e.g., ten minutes, etc.) in the proximity of the selected emergency response facility 108. In some such examples, when the emergency router 122 determines that the emergency request was sent in bad faith, the emergency router 122 forwards details (e.g., time of day, identifier associated with the autonomous vehicle 106, the route of the emergency corridor 102, etc.) to a regulatory and/or law enforcement authority.

The selected route is based on, for example, weather data, traffic data, the locations of infrastructure nodes 110a and 110b, and/or other advisories (e.g., road closures, other emergency corridors, etc.), etc. The emergency router 122 provides the selected route to the autonomy unit 112 of the autonomous vehicle 106. Additionally, the emergency router 122 determines which ones of the infrastructure nodes 110a and 110b are along the selected route of the emergency corridor 102. The emergency router 122 instructs the infrastructure nodes 110a are along the selected route to broad cast the emergency corridor message, which includes the current location of the autonomous vehicle 106, the velocity of the autonomous vehicle 106, the route of the emergency corridor 102, and a requested lane to move out of. From time to time, the emergency router 122 updates the emergency corridor message to reflect the current location of the autonomous vehicle 106, the current velocity of the autonomous vehicle 106, and/or any changes to the route of the emergency corridor 102.

The emergency router 122 determines whether to authorize the autonomous vehicle 106 to engage in the emergency mode. The determination is based on, for example, the weather, road conditions, and/or the traffic. For example, if the roads are icy, the emergency router 122 may not authorize the autonomous vehicle 106 to engage in the emergency mode. In some examples, the emergency router 122 may instruct the autonomous vehicle to “stop and wait.” In response to receiving the instruction to stop and wait, the autonomous vehicle 106 maneuvers to a side of the road (e.g., the right lane, a street parking area, a shoulder, etc.). In some such examples, the emergency router 122 instructs an emergency vehicle (e.g., an ambulance, a fire truck, a police vehicle, etc.) to the location at which the autonomous vehicle 106 is waiting.

FIG. 2 is a block diagram of the emergency dispatch server 104 of FIG. 1. In the illustrated example, the emergency dispatch server 104 is communicatively coupled to the infrastructure nodes 110a and 110b via wireless network infrastructure 202. The wireless network infrastructure 202 (a) manages the connection between the emergency dispatch server 104 and the infrastructure nodes 110a and 110b and (b) routes instructions and information between the emergency dispatch server 104 and the infrastructure nodes 110a and 110b. The wireless network infrastructure 202 may include one or more of a wide area network (e.g., such as a cellular network, a satellite communication network, WiMAX, and/or local area network(s) (e.g., IEEE 802.11 a/b/g/n/ac, etc.).

The emergency dispatch server 104 includes a dispatch module 204, a node database 206, an emergency coordinator module 208, and the emergency router 122. The dispatch module 204 is communicatively coupled with the autonomous vehicle 106 via the infrastructure nodes 110a and 110b and/or a cellular network. The dispatch module 204 receives the location of the autonomous vehicle 106 included in the emergency request. Additionally, the dispatch module 204 tracks the locations of the autonomous vehicle 106. In some examples, the autonomous vehicle 106, from time-to-time (e.g., periodically, aperiodically, etc.), sends its current location to the emergency dispatch server 104.

The node database 206 stores the coordinates of the infrastructure nodes 110a and 110b. In some examples, the node database 206 includes information regarding properties of the infrastructure nodes 110a and 110b, such as directionality, maintenance history, approximate range, nearby intersections, etc. The node database 206 may be implemented using any suitable memory and/or data storage apparatus and techniques.

The emergency coordinator module 208 is communicatively coupled to the emergency response facility 108. When the emergency router 122 selects one of the potential emergency response facilities 108 in response to receiving the emergency request, the emergency coordinator module 208 sends a message to the selected emergency response facility 108 that includes the ETA, and the nature of the emergency. Additionally, the emergency coordinator module 208 follows up with the emergency response facility 108 to determine the actual time of arrival and/or whether the occupant is being treated. If the autonomous vehicle 106 does not arrive and/or the occupant is not in need of treatment, the emergency coordinator module 208 may flag the autonomous vehicle 106 for further investigation and/or report details of the incident to a regulatory or law enforcement authority. In some examples, the emergency coordinator module 208 receives status messages from the emergency response facility 108 with the availability of the corresponding emergency response facility 108.

The emergency router 122 is commutatively coupled to the infrastructure nodes 110a and 110b via the wireless network infrastructure 202. The emergency router 122 is communicatively connected to a weather server 210 that provides weather data, a traffic server 212 that provides traffic data, and a navigation server 214 that provides map and navigation data (e.g., road composition, road grade, curves, etc.). In some examples, the servers 210, 212, and 214 provide application program interfaces (APIs) to facilitate the emergency router 122 obtaining the corresponding data.

The emergency router 122 receives the location of the autonomous vehicle 106 from the dispatch module 204. The emergency router 122 selects one of the potential emergency response locations based on (i) the distance between potential emergency response facilities 108 and the autonomous vehicle 106, (ii) preferences of the occupants that is experiencing the emergency (e.g., from a profile stored by the autonomous vehicle 106 and/or the emergency dispatch server 104), (iii) estimated travel time to the potential emergency response facilities 108, (iv) the nature of the emergency, (v) specialties of the potential emergency response facilities 108, and/or (vi) availability of the potential emergency response facilities 108, etc. After selecting the emergency response facility 108, the emergency router 122 determines potential routes between the location of the autonomous vehicle 106 and the selected emergency response facility 108. The potential routes are divided into segments. For example, the segments may represent a portion of road between two intersections. The emergency router 122 analyzes the segments based on the weather data, the traffic data, and/or the navigation data to select a contiguous set of segments from the location of the autonomous vehicle 106 to the emergency response facility 108 to the route of the emergency corridor 102.

Based on the route of the emergency corridor 102, the emergency router 122 receives identifiers (e.g., network addresses, etc.) of the infrastructure nodes 110a along the route from the node database 206. The emergency router 122 generates an emergency message and instructs the identified infrastructure nodes 110a to broadcast the emergency message. The emergency router 122 sends the route of the emergency corridor 102 to the autonomy unit 112 of the autonomous vehicle 106. In some examples, the emergency router 122 sends the emergency message to the autonomous vehicle 106 for the autonomous vehicle 106 to broadcast while traveling to the emergency response facility 108.

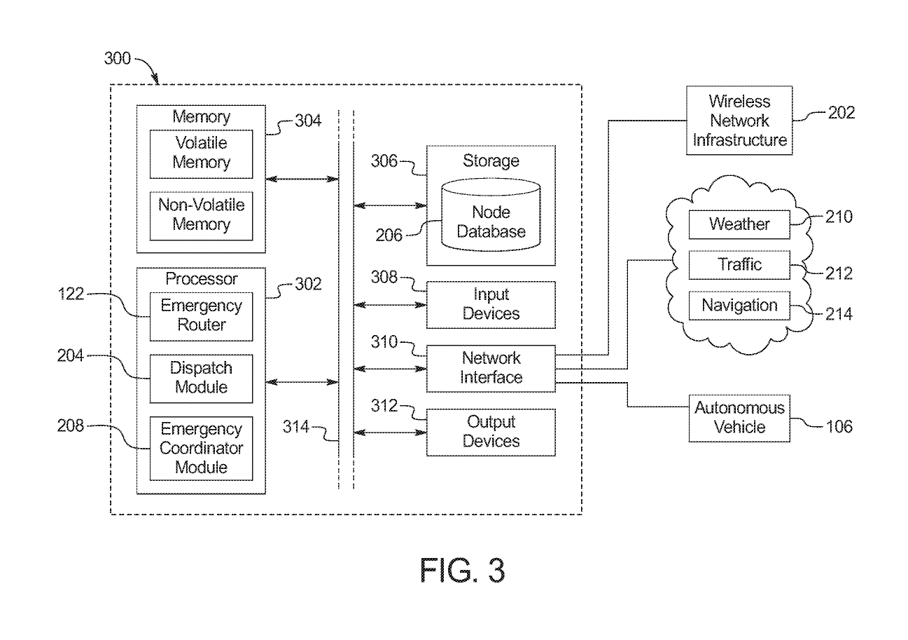

FIG. 3 is a block diagram of electronic components 300 of the emergency dispatch server 104 of FIGS. 1 and 2. In the illustrated example, the electronic components 300 include a processor or controller 302, memory 304, storage 306, a network interface 308, input devices 310, output devices 312, and a data bus 314.

The processor or controller 302 may be any suitable processing device or set of processing devices such as, but not limited to: a microprocessor, a microcontroller-based platform, a suitable integrated circuit, or one or more application-specific integrated circuits (ASICs). In the illustrated example, the processor or controller 302 is structured to include the dispatch module 204, the emergency coordinator module 208, and the emergency router 122. The memory 304 may be volatile memory (e.g., RAM, which can include non-volatile RAM, magnetic RAM, ferroelectric RAM, and any other suitable forms); non-volatile memory (e.g., disk memory, FLASH memory, EPROMs, EEPROMs, memristor-based non-volatile solid-state memory, etc.), unalterable memory (e.g., EPROMs), and read-only memory. In some examples, the memory 304 includes multiple kinds of memory, particularly volatile memory and non-volatile memory. The storage 306 may include any high-capacity storage device, such as a hard drive, and/or a solid state drive. In the illustrated example, the node database 206 is stored in the storage 306.

The memory 304 and the storage 306 are a computer readable medium on which one or more sets of instructions, such as the software for operating the methods of the present disclosure can be embedded. The instructions may embody one or more of the methods or logic as described herein. In a particular embodiment, the instructions may reside completely, or at least partially, within any one or more of the memory 304, the computer readable medium, and/or within the processor 302 during execution of the instructions.

The terms “non-transitory computer-readable medium” and “computer-readable medium” should be understood to include a single medium or multiple media, such as a centralized or distributed database, and/or associated caches and servers that store one or more sets of instructions. The terms “non-transitory computer-readable medium” and “computer-readable medium” also include any tangible medium that is capable of storing, encoding or carrying a set of instructions for execution by a processor, or that cause a system to perform any one or more of the methods or operations disclosed herein. As used herein, the term “computer readable medium” is expressly defined to include any type of computer readable storage device and/or storage disk and to exclude propagating signals.

The network interface 308 facilitates the emergency dispatch server 104 communicating with other network devices. The network interface 308 includes a communication device, such as a modem or a network interface card, to facilitate exchange of data with the wireless network infrastructure 202, the weather server 210, the traffic server 212, the navigation server 214, and/or the autonomous vehicle 106 (e.g., an Ethernet connection, a digital subscriber line (DSL), a telephone line, coaxial cable, a cellular telephone system, etc.).

The input device(s) 310 facilitate a user interacting with the electronic components 300. The input device(s) 310 can be implemented by, for example, a serial port, a Universal Serial Bus (USB) port, a IEEE 1339 port, a keyboard, a button, a mouse, a touchscreen, a track-pad, and/or a voice recognition system. The output device(s) 312 facilitate the electronic components 300 providing information to the user. The output devices 312 can be implemented, for example, by display devices (e.g., a light emitting diode (LED), an organic light emitting diode (OLED), a liquid crystal display, a cathode ray tube display (CRT), a touchscreen, etc.), and/or communication devices (the serial port, the USB port, the IEEE 1339 port, etc.).

The data bus 314 communicatively couples the processor 302, the memory 304, the storage 306, the network interface 308, the input devices 310, and the output devices 312. The data bus 314 may be implemented by one or more interface standards, such as an Ethernet interface, a USB interface, PCI express interface, and/or a Serial ATA interface, etc.

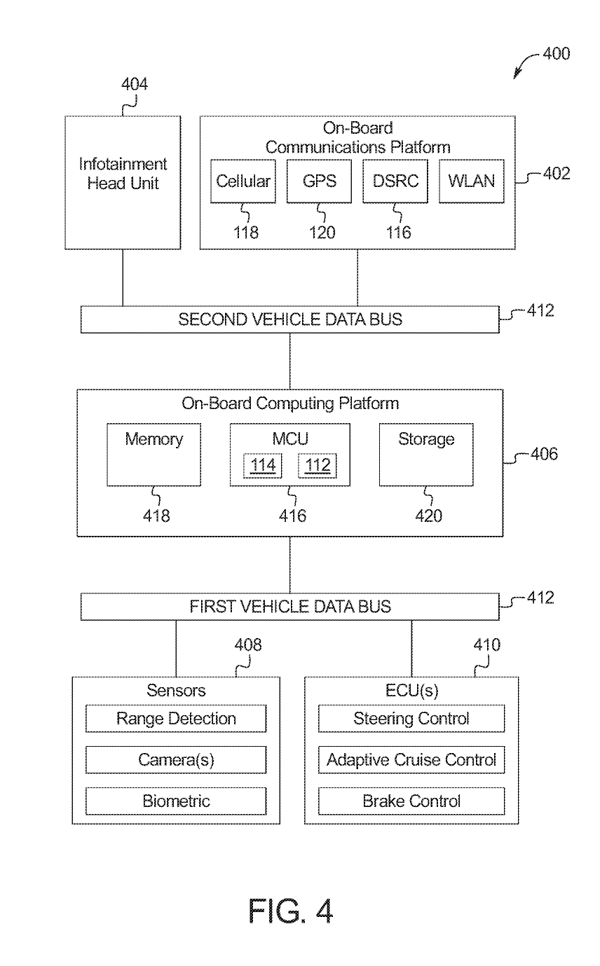

FIG. 4 is a block diagram of electronic components 400 of the autonomous vehicle 106 of FIG. 1. The electronic components 400 include an example on-board communications platform 402, the example infotainment head unit 404, an on-board computing platform 406, example sensors 408, example electronic control units (ECUs) 410, a first vehicle data bus 412, and second vehicle data bus 414.

The on-board communications platform 402 includes wired or wireless network interfaces to enable communication with external networks. The on-board communications platform 402 also includes hardware (e.g., processors, memory, storage, antenna, etc.) and software to control the wired or wireless network interfaces. In the illustrated example, the on-board communications platform 402 includes the DSRC module 116, the cellular modem 118, and the GPS receiver 120. The on-board communications platform 402 may also include one or more controllers for wireless local area networks such as a Wi-FI® controller (including IEEE 802.11 a/b/g/n/ac or others), a Bluetooth® controller (based on the Bluetooth® Core Specification maintained by the Bluetooth Special Interest Group), and/or a ZigBee® controller (IEEE 802.15.4), and/or a Near Field Communication (NFC) controller, etc. Additionally, the on-board communications platform 402 may also include a wired interface (e.g. an auxiliary port, etc.) to enable direct communication with an electronic device (such as, a smart phone, a tablet computer, a laptop, etc.).

The infotainment head unit 404 provides an interface between the autonomous vehicle 106 and a user (e.g., a driver, a passenger, etc.). The infotainment head unit 404 includes digital and/or analog interfaces (e.g., input devices and output devices) to receive input from the user(s) and display information. The input devices may include, for example, a control knob, an instrument panel, a digital camera for image capture and/or visual command recognition, a touch screen, an audio input device (e.g., cabin microphone), buttons, or a touchpad. The output devices may include instrument cluster outputs (e.g., dials, lighting devices), actuators, a heads-up display, a center console display (e.g., a liquid crystal display (“LCD”), an organic light emitting diode (“OLED”) display, a flat panel display, a solid state display, etc.), and/or speakers. The infotainment head unit 404 may include an emergency request button to facilitate the occupant(s) of the autonomous vehicle 106 sending the emergency request to the emergency dispatch server 104.

The on-board computing platform 406 includes a processor or controller 416, memory 418, and storage 420. In some examples, the on-board computing platform 406 is structured to include the autonomy unit 112 and/or the health monitoring unit 114. The processor or controller 416 may be any suitable processing device or set of processing devices such as, but not limited to: a microprocessor, a microcontroller-based platform, a suitable integrated circuit, one or more FPGAs, and/or one or more ASICs. The memory 418 may be volatile memory (e.g., RAM, which can include non-volatile RAM, magnetic RAM, ferroelectric RAM, and any other suitable forms); non-volatile memory (e.g., disk memory, FLASH memory, EPROMs, EEPROMs, memristor-based non-volatile solid-state memory, etc.), unalterable memory (e.g., EPROMs), and read-only memory. In some examples, the memory 418 includes multiple kinds of memory, particularly volatile memory and non-volatile memory. The storage 420 may include any high-capacity storage device, such as a hard drive, and/or a solid state drive.

The memory 418 and the storage 420 are a computer readable medium on which one or more sets of instructions, such as the software for operating the methods of the present disclosure can be embedded. The instructions may embody one or more of the methods or logic as described herein. In a particular embodiment, the instructions may reside completely, or at least partially, within any one or more of the memory 418, the computer readable medium, and/or within the processor 416 during execution of the instructions.

The sensors 408 may be arranged in and around the autonomous vehicle 106 in any suitable fashion. In the illustrated example, the sensors 408 include range detection sensors and camera(s). The ECUs 410 monitor and control the systems of the autonomous vehicle 106. The ECUs 410 communicate and exchange information via the first vehicle data bus 412. Additionally, the ECUs 410 may communicate properties (such as, status of the ECU 410, sensor readings, control state, error and diagnostic codes, etc.) to and/or receive requests from other ECUs 410. Some autonomous vehicles 106 may have seventy or more ECUs 410 located in various locations around the autonomous vehicles 106 communicatively coupled by the first vehicle data bus 412. The ECUs 410 are discrete sets of electronics that include their own circuit(s) (such as integrated circuits, microprocessors, memory, storage, etc.) and firmware, sensors, actuators, and/or mounting hardware. In the illustrated example, the ECUs 410 include the steering control unit, the adaptive cruise control unit, and the brake control unit.

The first vehicle data bus 412 communicatively couples the sensors 408, the ECUs 410, the on-board computing platform 406, and other devices connected to the first vehicle data bus 412. In some examples, the first vehicle data bus 412 is implemented in accordance with the controller area network (CAN) bus protocol as defined by International Standards Organization (ISO) 11898-1. Alternatively, in some examples, the first vehicle data bus 412 may be a Media Oriented Systems Transport (MOST) bus, or a CAN flexible data (CAN-FD) bus (ISO 11898-7). The second vehicle data bus 414 communicatively couples the on-board communications platform 402 the infotainment head unit 404, and the on-board computing platform 406. The second vehicle data bus 414 may be a MOST bus, a CAN-FD bus, or an Ethernet bus. In some examples, the on-board computing platform 406 communicatively isolates the first vehicle data bus 412 and the second vehicle data bus 414 (e.g., via firewalls, message brokers, etc.). Alternatively, in some examples, the first vehicle data bus 412 and the second vehicle data bus 414 are the same data bus.

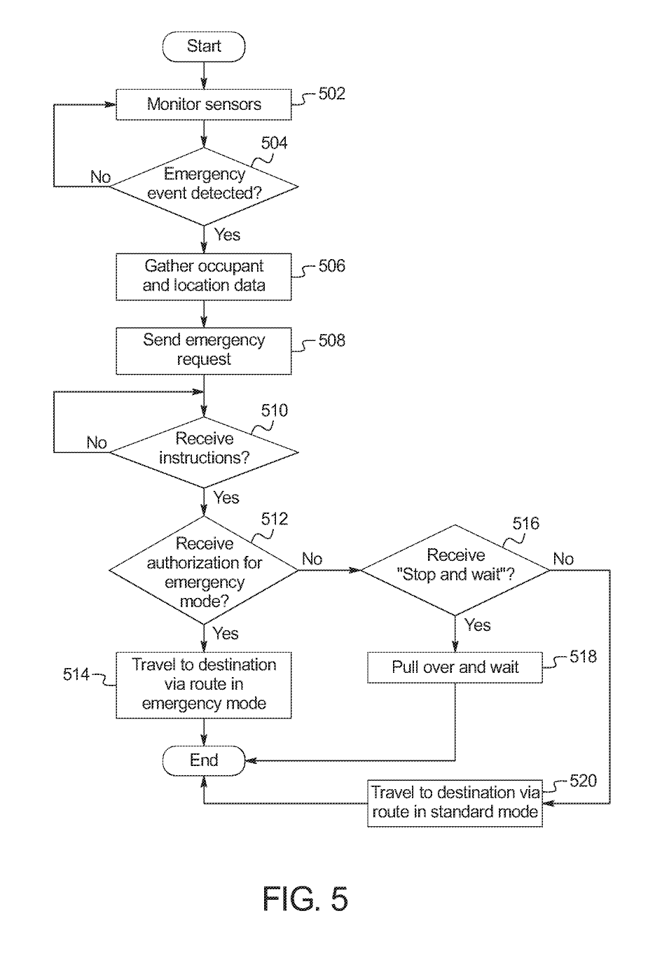

FIG. 5 is a flowchart of an example method to override autonomous behavior of the autonomous vehicle 106 of FIG. 1. Initially, at block 502, the health monitoring unit 114 monitors the sensors (e.g. the sensors 408 of FIG. 4) of the autonomous vehicle 106. At block 504, the health monitoring unit 114 determines whether an occupant of the autonomous vehicle 106 is experiencing an emergency based on the sensors monitored at block 502. In some examples, the health monitoring unit 114 maintains a table or other data structure that specifies sensor values that at indicative of various emergency conditions. If the occupant of the autonomous vehicle 106 is experiencing an emergency, the method continues at block 506. Otherwise, the occupant of the autonomous vehicle 106 is not experiencing an emergency, the method returns to block 502. At block 506, the health monitoring unit 114 gathers the coordinates of the autonomous vehicle 106 (e.g., from the GPS receiver 120) and information about the occupants of the autonomous vehicle 106. In some examples, the health monitoring unit 114 gathers information about the occupants of the autonomous vehicle 106, such as the quantity of occupants, the locations of the occupants inside the autonomous vehicle 106, and/or the identity of the occupant experiencing the emergency, etc. At block 508, the health monitoring unit 114, via the DSRC module 116 and/or the cellular modem 118, sends the emergency request to the emergency dispatch server 104. In some examples, the health monitoring unit 114 includes the health data collected by the biometric sensors with the emergency request.

At block 510, the autonomy unit 112 waits until receiving instructions from the emergency dispatch server 104. At block 512, the autonomy unit 112 determines whether the instructions from the emergency dispatch server 104 include an authorization to use the emergency mode. If the instructions from the emergency dispatch server 104 include an authorization to use the emergency mode, the method continues at block 514. Otherwise, if the instructions from the emergency dispatch server 104 do not include an authorization to use the emergency mode, the method continues at block 516. At block 514, the autonomy unit 112 operates the autonomous vehicle to travel the route of the emergency corridor 102 using the emergency mode. While traveling through the emergency corridor 102, the autonomy unit 112 sends messages to the emergency dispatch server 104 with the current location, the current speed, and the current heading of the autonomous vehicle 106.

At block 516, the autonomy unit 112 determines whether the instructions from the emergency dispatch server 104 include instructions to “stop and wait.” If the instructions from the emergency dispatch server 104 include instructions to “stop and wait,” the method continues to block 518. Otherwise, if the instructions from the emergency dispatch server 104 do not include instructions to “stop and wait,” the method continues to block 520. At block 518, the autonomy unit 112 operates the autonomous vehicle 106 to pull over and stop. In some examples, after the autonomous vehicle 106 stops, the autonomy unit 112, from time-to-time, broadcasts the emergency request to the emergency dispatch server 104. At block 520, the autonomy unit 112 operates the autonomous vehicle 106 to travel the route of the emergency corridor 102 using the standard mode. While traveling through the emergency corridor 102, the autonomy unit 112 sends messages to the emergency dispatch server 104 with the current location, the current speed, and the current heading of the autonomous vehicle 106.

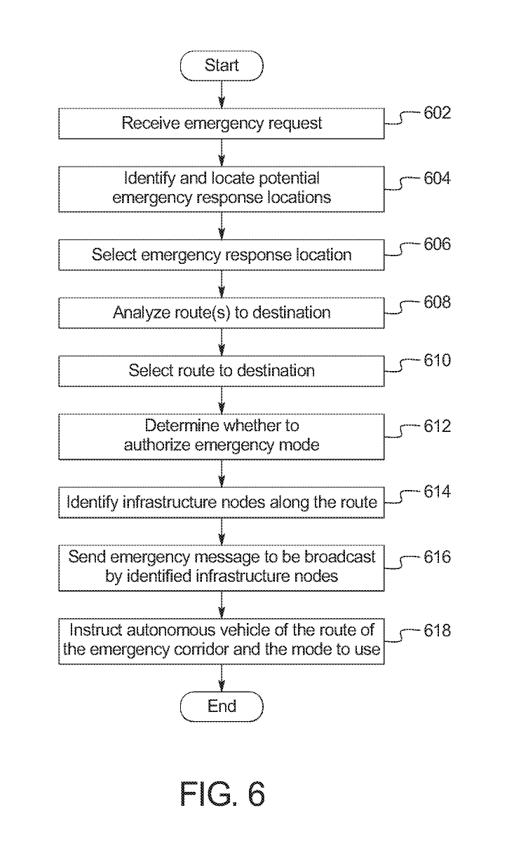

FIG. 6 is a flowchart of an example method to create the emergency corridor 102 of FIG. 1. Initially, at block 602, the dispatch module 204 receives the emergency request from the autonomous vehicle 106. At block 604, the emergency router 122 identifies and locates the potential emergency response facilities 108. At block 606, the emergency router 122 selects one of the potential emergency response facilities 108 identified at block 604. At block 608, the emergency router 122 analyzes the route(s) between the autonomous vehicle 106 and the emergency response facility 108 selected at block 606. At block 610, the emergency router 122 selects the route to the emergency response facility 108 to become the emergency corridor 102. At block 612, the emergency router 122 determines whether to authorized emergency mode for the autonomy unit 112 of the autonomous vehicle 106. At block 614, the emergency router 122 identifies the infrastructure nodes 110a along the route selected at block 610. At block 616, the emergency router 122 instructs the infrastructure nodes 110a identified at block 614 to broadcast an emergency corridor message including the current location of the autonomous vehicle 106, the velocity of the autonomous vehicle 106, the route of the emergency corridor 102, and instructions (e.g., which lane to clear, etc.). At block 618, the emergency router 122 sends the route of the emergency corridor 102 to the autonomous vehicle 106 and informs the autonomous vehicle 106 of which mode it is to use to traverse the emergency corridor 102. The method of FIG. 6 then ends.

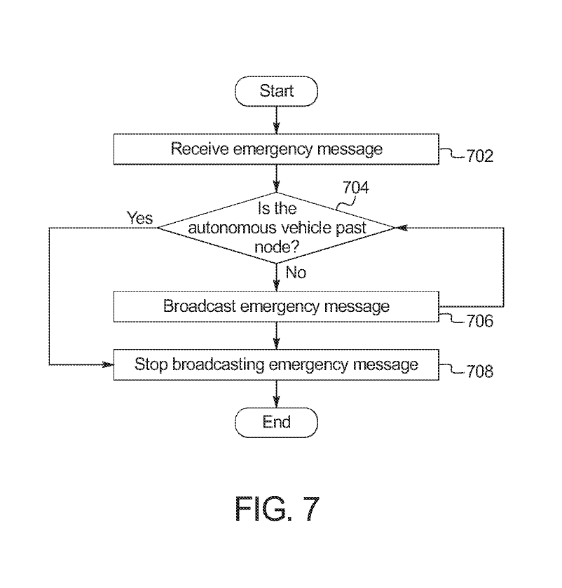

FIG. 7 is a flowchart of an example method to broadcast emergency messages by infrastructure nodes 110a along the route of the emergency corridor 102. Initially, at block 702, the infrastructure node 110a receives the instruction to broadcast the emergency corridor message from the emergency dispatch server 104. At block 704, the infrastructure node 110a determines whether the autonomous vehicle 106 has passed the infrastructure nodes 110a based on the location of the infrastructure node 110a, the current location of the autonomous vehicle 106 included in the instructions to broadcast the emergency corridor message, and the route of the emergency corridor 102 included in the instructions to broadcast the emergency corridor message. If the infrastructure node 110a determines the autonomous vehicle 106 has not passed the infrastructure node 110a, at block 706, the infrastructure node 110a broadcasts the emergency corridor message. Otherwise, if the infrastructure node 110a determines the autonomous vehicle 106 has passed the infrastructure node 110a, at block 708, the infrastructure node 110a ends broadcasting the emergency corridor message. The method of FIG. 7 then ends.

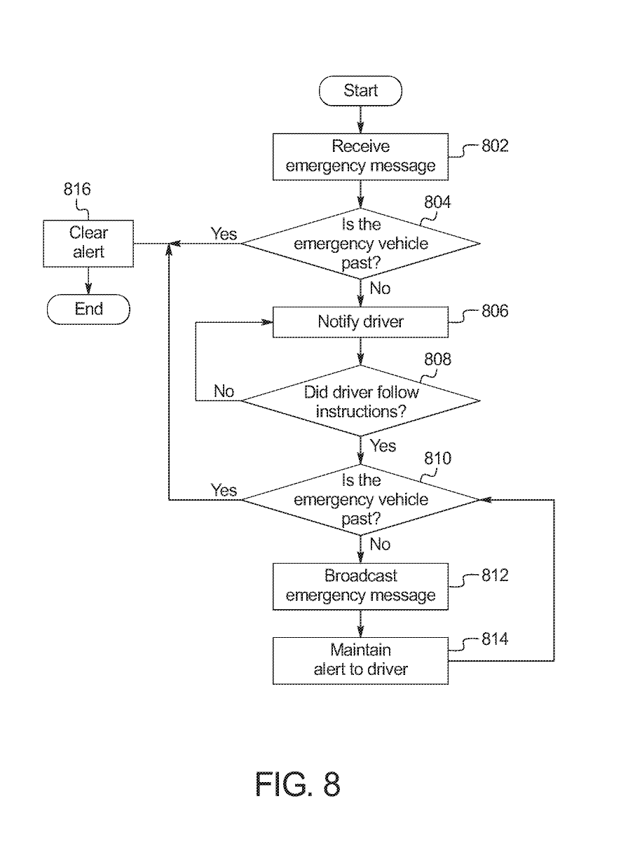

FIG. 8 is a flowchart of an example method for vehicles to react to the emergency messages broadcast by the infrastructure nodes 110a along the route of the emergency corridor 102. Initially, at block 802, the vehicle receives the emergency corridor message. At block 804, the vehicle determines whether the autonomous vehicle 106 has passed the vehicle based on the location of the vehicle, the current location of the autonomous vehicle 106 included in the emergency corridor message, and the route of the emergency corridor 102 included in the emergency corridor message. If the autonomous vehicle 106 has passed the vehicle, the method continues at block 816. Otherwise, if the autonomous vehicle 106 has not passed the vehicle, the method continues at block 806.

At block 806, the vehicle notifies its occupants of the autonomous vehicle 106 traversing the emergency corridor 102. The vehicle provides a visual and/or audible alert via a dashboard display and/or the speakers of the vehicle based on instructions in the emergency corridor message. At block 808, the vehicle determines whether the driver followed the instructions. For example, the vehicle may analyze the output of the steering control unit to determine whether the vehicle moved to the right, or analyze the output of the brake control unit to determine whether the vehicle stopped. If the driver did not follow the instructions, the method returns to block 806, at which the vehicle notifies the driver. In some examples, the vehicle escalates the level of notification. If the driver followed the instructions, the method continues to block 810.

At block 810, the vehicle determines whether the autonomous vehicle 106 has passed the vehicle based on the location of the vehicle, the current location of the autonomous vehicle 106 included in the emergency corridor message, and the route of the emergency corridor 102 included in the emergency corridor message. If the autonomous vehicle 106 has passed the vehicle, the method continues at block 816. Otherwise, if the autonomous vehicle 106 has not passed the vehicle, the method continues at block 812. At block 812, the vehicle rebroadcasts the emergency corridor message. At block 814, the vehicle continues to notify the driver. In some examples, the notification may change to, for example, just a visual notification on the dashboard display. At block 816, the vehicle clears the notification(s) of the emergency corridor message and/or discontinues broadcasting the emergency notification message.

The flowchart of FIG. 5 is a method that may be implemented by machine readable instructions that comprise one or more programs that, when executed by a processor (such as the processor 416 of FIG. 4), cause the autonomous vehicle 106 to implement the autonomy unit 112 and/or the health monitoring unit 114 of FIG. 1. The flowchart of FIG. 6 is a method that may be implemented by machine readable instructions that comprise one or more programs that, when executed by a processor (such as the processor 302 of FIG. 3), cause the emergency dispatch server 104 to implement the emergency router 122 of FIGS. 1, 2, and 3. The flowchart of FIG. 8 is a method that may be implemented by machine readable instructions that comprise one or more programs that, when executed by a processor, implement the infrastructure nodes 110a and 110b of FIG. 1. The flowchart of FIG. 9 is a method that may be implemented by machine readable instructions that comprise one or more programs that, when executed by a processor, implement vehicles creating the emergency corridor 102. Further, although the example program(s) is/are described with reference to the flowcharts illustrated in FIGS. 5, 6, 7, and 8, many other methods of implementing the example autonomy unit 112, the example health monitoring unit 114 the example emergency router 122, and/or the infrastructure nodes 110a and 110b may alternatively be used. For example, the order of execution of the blocks may be changed, and/or some of the blocks described may be changed, eliminated, or combined.

In this application, the use of the disjunctive is intended to include the conjunctive. The use of definite or indefinite articles is not intended to indicate cardinality. In particular, a reference to “the” object or “a” and “an” object is intended to denote also one of a possible plurality of such objects. Further, the conjunction “or” may be used to convey features that are simultaneously present instead of mutually exclusive alternatives. In other words, the conjunction “or” should be understood to include “and/or.” The terms “includes,” “including,” and “include” are inclusive and have the same scope as “comprises,” “comprising,” and “comprise” respectively.

The above-described embodiments, and particularly any “preferred” embodiments, are possible examples of implementations and merely set forth for a clear understanding of the principles of the invention. Many variations and modifications may be made to the above-described embodiment(s) without substantially departing from the spirit and principles of the techniques described herein. All modifications are intended to be included herein within the scope of this disclosure and protected by the following claims.

What is claimed is:

1. An emergency response system comprising:
an autonomous vehicle to send an emergency request in response to detecting an occupant experiencing a medical emergency;
infrastructure nodes distributed across a municipal area; and
an emergency router to:
select a route from a first location of the autonomous vehicle to an emergency response facility;
select the infrastructure nodes that are along the route; and
instruct the selected infrastructure nodes to broadcast messages to create an emergency corridor.

2. The system of claim 1, wherein the messages include the route of the emergency corridor, and a current location, a current heading, and a current speed of the autonomous vehicle.

3. The system of claim 1, wherein the emergency router is to:
determine whether to authorize the autonomous vehicle to operate in an emergency mode;
instruct the autonomous vehicle to traverse the route; and
periodically update the messages to include a current location, current heading, and a current speed of the autonomous vehicle.

4. The system of claim 1, wherein the selected ones of the infrastructure nodes are to determine when to stop broadcasting the messages based on second locations of the selected ones of the infrastructure nodes and a current location of the autonomous vehicle identified in the messages.

5. The system of claim 1, wherein the autonomous vehicle is to broadcast the messages.

6. The system of claim 1, wherein the emergency router is to select the emergency response facility from a plurality of emergency response facilities.

7. The system of claim 6, wherein the emergency router is to select the emergency response facility based on at least one of (i) distances between the plurality of emergency response facilities and the autonomous vehicles, (ii) preferences of the occupant of the autonomous vehicle, (iii) estimated travel times to the plurality of emergency response facilities, (iv) a type of the medical emergency, (v) specialties of the plurality of emergency response facilities, or (vi) availabilities of the plurality of emergency response facilities.

8. A method to create an emergency corridor for an autonomous vehicle, the method comprising:
receiving an emergency request from the autonomous vehicle;
selecting, via a processor, an emergency facility;
determining a route from the autonomous vehicle to the emergency facility;
determining infrastructure nodes along the route; and
broadcasting emergency messages from the infrastructure nodes along the route, the emergency messages including the route, a location, a heading, and a speed of the autonomous vehicle.

9. The method of claim 8, including:
determining whether to authorize the autonomous vehicle to operate in an emergency mode;
instructing the autonomous vehicle to traverse the route; and
periodically updating the emergency messages to include a current location, a current heading, and a current speed of the autonomous vehicle.

10. The method of claim 8, wherein determining the route for the autonomous vehicle includes analyzing traffic data and weather data between a first location of the autonomous vehicle and a second location of the emergency facility.

11. The method of claim 8, wherein the emergency messages cause a vehicle to:
determine whether the vehicle is traveling on the route or will intersect the route specified by the emergency message; and
in response to determining that the vehicle is traveling on the route or will intersect the route, provide audio and visual instructions to clear a lane for the emergency vehicle.

12. The method of claim 8, wherein selecting the emergency response facility is based on at least one of (i) distances between a plurality of emergency response facilities and the autonomous vehicles, (ii) preferences of an occupant of the autonomous vehicle, (iii) estimated travel times to the plurality of emergency response facilities, (iv) a medical emergency specified by the emergency request, (v) specialties of the plurality of emergency response facilities, or (vi) availabilities of the plurality of emergency response facilities.

13. The method of claim 8, wherein the emergency request includes health data collected by biometric sensors of the autonomous vehicle, and wherein after selecting the emergency facility, sending the health data to the selected emergency facility.

14. A method comprising:
monitoring biometric sensors in an autonomous vehicle to detect when an occupant is experiencing a medical emergency;
in response to detecting the medical emergency, sending an emergency request to an emergency dispatch server; and
in response to receiving instructions from the emergency dispatch server, operating, by a processor, the autonomous vehicle to follow a route specified by the instructions.

15. The method of claim 14, including determining a mode specified by the instructions.

16. The method of claim 15, wherein the mode specified by the instructions is one of a standard mode or an emergency mode.

17. The method of claim 14, including broadcasting, via a DSRC module, an emergency message received from the emergency dispatch server.

18. The method of claim 14, including periodically sending an update to the emergency dispatch server including a current location, a current heading, and a current speed of the autonomous vehicle.

19. The method of claim 14, wherein the emergency request includes health data collected by the biometric sensors.

20. The method of claim 14, including, in response to determining the emergency request was sent to the emergency dispatch server in bad faith, reporting an identifier associated with the autonomous vehicle to a law enforcement authority.
